(19) United States
(12) Patent Application Publication
(10) Pub. No.: US 2023/0025377 A1
(43) Pub. Date: Jan. 26, 2023

(54) COMPOSITIONS AND METHODS FOR TREATING OR PREVENTING PRURITUS

(71) Applicant: HLB THERAPEUTICS CO., LTD., Gyeonggi-do (KR)

(72) Inventors: Sin Wook KANG, Gyeonggi-do (KR); Ji Hye SUNG, Seoul (KR); Won Suk YANG, Princeton, NJ (US)

(73) Assignee: HLB THERAPEUTICS CO., LTD., Gyeonggi-do (KR)

(21) Appl. No.: 17/797,643

(22) PCT Filed: Feb. 12, 2021
(86) PCT No.: PCT/US2021/017910

§ 371 Date: Aug. 4, 2022

Related U.S. Application Data
(60) Provisional application No. 62/976,060, filed on Feb. 13, 2020.

(51) Int. Cl. A61K 38/22; A61K 9/06; A61P 17/00

(52) U.S. Cl. CPC A61K 38/2292; A61K 9/06; A61P 17/00

(57) ABSTRACT

A method of treating or preventing pruritus in a subject in need thereof using a pharmaceutical composition is provided. The method includes administering a therapeutically effective amount of Tβ4, Tβ4 fragments, Tβ4 isoforms, Tβ4 derivatives, or variants thereof, wherein the therapeutically effective amount decreases release of a pruritus-related factor or decreases release of an inflammatory cytokine from a target tissue is provided.

### CROSS-REFERENCE TO RELATED APPLICATIONS

This application claims priority to U.S. Provisional Patent Application No. 62/976,060, filed on Feb. 13, 2020, the entire contents of which are incorporated by reference herein.

### FIELD OF THE INVENTION

The presently disclosed subject matter relates to methods and compositions for treating or preventing pruritus and the symptoms associated with the same. Specifically, the presently disclosed subject matter relates to methods and compositions for decreasing the release of pruritus-related factors from the target tissue.

### SEQUENCE LISTING

The specification further incorporates by reference the Sequence Listing submitted herewith via EFS on Feb. 12, 2021. Pursuant to 37 C.F.R. § 1.52(e)(5), the Sequence Listing text file, identified as Sequence Listing 086570.0104, is 453 bytes and was created on Jan. 11, 2021. The Sequence Listing, electronically filed herewith, does not extend beyond the scope of the specification and thus does not contain new matter.

### BACKGROUND

Exposure to chemical and biological hazards caused by changes in a residential environment, dietary changes and genetic influences, and environmental pollution due to industrialization and urbanization can increase allergic diseases in various age groups including childhood. Allergies can occur when a person reacts to certain external environments, which are harmless to most people. Certain people can experience a severe allergic reaction, such as respiratory diseases (e.g., asthma, rhinitis, chronic dermatitis, and atopy) that can affect the mental state and daily life of the patient.

Pruritus refers to symptoms associated with itchiness, which can be accompanied by an unpleasant sensation that causes the desire to scratch or rub the skin, eye, and/or nose. Certain nerve cells can perceive certain stimuli from tissues (e.g., skin, nose, and eye) and relay the stimuli to other nerves (e.g., central nervous system) and to cells (e.g., epithelial cells and immune cells), which can induce responses to the stimuli (itchiness). Pruritus symptoms can be systemic or can be targeted where specific locations are affected. Pruritus can be caused by contact with toxic sources including those from animals and plants, psoriasis, seborrheic dermatitis, and fungal growth, but it can sometimes be caused by allergic reactions to non-toxic substances.

Notably, pruritus can be multifactorial. Among other factors, allergy can cause severe pruritus. For example, people with allergies (e.g., atopic dermatitis) can be more prone to develop pruritus. Allergy involves a distinct biological reaction to a specific foreign substance, and the cause of the allergy can be a physiological pathological process resulting from an antigen-antibody reaction. An allergic reaction is generally classified as type 1-4 according to the time required to trigger a response and the microfibrillar complement-mediated pathogenic nature of the response. Immediate anaphylaxis, which can cause bronchial asthma, allergic rhinitis, hay fever, and atopic dermatitis, is known as a type 1 reaction. Immediate hypersensitivity and allergic reactions can be caused by various changes in mast cells.

Activated mast cells are one of the important players that are involved in allergic reactions and neurogenic inflammation. Mast cells can be activated by allergic reaction factors such as immunoglobulin E (IgE), histamine, leukotrien, and prostaglandin E2 (PGE2), and can cause allergic inflammation. Certain inflammatory cytokines can stimulate immune cells (e.g., T helper type 2 cells and B cells) and increase the release of IgE from the immune cells leading to the activation of mast cells. For example, interleukin 4 (IL-4), interleukin 13 (IL-13), and tumor necrosis factor α (TNF-α) can cause an inflammatory allergic response by increasing IgE secretion from immune cells. As such, there remains a need in the art for improved compositions for treating pruritus by reducing both the factors involved in the allergic reaction and the inflammatory cytokines.

### SUMMARY

The present disclosure provides pharmaceutical compositions and methods for treating or preventing pruritus. The method for treating or preventing pruritus in a subject in need thereof can include administering a therapeutically effective amount of thymosin beta 4 (Tβ4), Tβ4 fragments, Tβ4 isoforms, Tβ4 derivatives, or variants thereof, wherein the therapeutically effective amount decreases release of a pruritus-related factor or decreases release of an inflammatory cytokine from a target tissue.

In certain embodiments, the target tissue can be selected from the group consisting of skin, connective tissue, nerve, soft tissue, epithelial tissue, and combinations thereof. In non-limiting embodiments, the epithelial tissue can be selected from the group consisting of a oral epithelial tissue, a pharyngeal epithelial tissue, a nasal epithelial tissue, an ocular epithelial tissue, a gastric epithelial tissue, and combinations thereof.

In certain embodiments, the pruritus-related factor can be an allergic reaction factor. In non-limiting embodiments, the allergic reaction factor can be selected from the group consisting of histamine, leukotriene β4 (LTB4), prostaglandin E2, and combinations thereof. In some embodiments, the release of the allergic reaction factor from the target tissue can be reduced by at least about 10%, about 20%, or about 30%.

In certain embodiments, the inflammatory cytokine can be selected from the group consisting of interleukin 4, interleukin 13, tumor necrosis factor α, and combinations thereof. In some embodiments, the release of the inflammatory cytokine from the target tissue can be reduced by at least about 10%, about 20%, about 30%, or about 40%.

In certain embodiments, the therapeutically effective amount of Tβ4, Tβ4 fragments, Tβ4 isoforms, Tβ4 derivatives, or variants thereof can be applied to the target tissue in a form of a solution, lotion, plaster, gel, cream, paste, spray, suspension, dispersion, hydrogel, ointment, oil, patch, lens, inhaler, nasal spray, or foaming agent. In non-limiting embodiments, the therapeutically effective amount of Tβ4, Tβ4 fragments, Tβ4 isoforms, Tβ4 derivatives, or variants thereof can be applied to the target tissue at least one time a day.

In certain embodiments, the therapeutically effective amount of Tβ4, Tβ4 fragments, Tβ4 isoforms, Tβ4 derivatives, or variants thereof can be from about 50 μg/ml to about 10 mg/ml.

In certain embodiments, the present disclosure provides a pharmaceutical composition for treating or preventing pruritus in a subject. The pharmaceutical composition can include a therapeutically effective amount of Tβ4, Tβ4 fragments, Tβ4 isoforms, Tβ4 derivatives, or variants thereof, wherein the therapeutically effective amount is present in an amount to decrease release of a pruritus-related factor or decrease release of an inflammatory cytokine from a target tissue.

In certain embodiments, the pruritus-related factor can be an allergic reaction factor. In non-limiting embodiments, the allergic reaction factor can be selected from the group consisting of histamine, leukotriene β4, prostaglandin E2, and combinations thereof.

In certain embodiments, the inflammatory cytokine can be selected from the group consisting of interleukin 4, interleukin 13, tumor necrosis factor α, and combinations thereof.

In certain embodiments, the pharmaceutical composition can be in a form of a solution, lotion, plaster, gel, cream, paste, spray, suspension, dispersion, hydrogel, ointment, oil, patch, lens, inhaler, nasal spray, or foaming agent.

In certain embodiments, the therapeutically effective amount of Tβ4, Tβ4 fragments, Tβ4 isoforms, Tβ4 derivatives, or variants thereof in the composition can be from about 50 μg/ml to about 10 mg/ml.

### BRIEF DESCRIPTION OF THE DRAWINGS

FIG. 1 provides a graph illustrating the effects of phorbol 12-myristate 13-acetate and calcium ionophore (PMACI) on histamine release in HMC-1 cells. Values represent the mean values±standard error (S.E.) of three independent experiments. ### represents the p-value is less than 0.001 (p<0.001) compared with the normal group (CON).

FIG. 2 provides a graph illustrating the inhibitory effects of thymosin beta 4 (Tβ4) at above about 1 μg/ml concentration on histamine release in HMC-1 cells. Values represent the mean values±S.E. of three independent experiments. *** represents that the p-value is less than 0.001 (p<0.001) compared with CON. ## represents that the p-value is less than 0.01 (p<0.01), compared with the PMACI treated group (EXP1). ### represents that the p-value is less than 0.001 (p<0.001), compared with EXP1.

FIG. 3 provides a graph illustrating the inhibitory effects of Tβ4 at above about 1.5 μg/ml concentration on leukotrien B4 (LTB4) release in PMACI-treated HMC-1 cells. Values represent the mean values±S.E. of three independent experiments. *** represents that the p-value is less than 0.001 (p<0.001) compared with CON. # represents that the p-value is less than 0.05 (p<0.05) compared with EXP1.

FIG. 4 provides a graph illustrating the inhibitory effects of Tβ4 at above about 3 μg/ml concentration on prostaglandin E2 (PGE2) release in PMACI-treated HMC-1 cells. Values represent the mean values±S.E. of three independent experiments. *** represents that the p-value is less than 0.001 (p<0.001) compared with CON. ## represents that the p-value is less than 0.01 (p<0.01) compared with EXP1. ### represents that the p-value is less than 0.001 (p<0.001) compared with EXP1.

FIG. 5 provides a graph illustrating the inhibitory effects of Tβ4 at above about 1.5 μg/ml concentration on interleukin 4 (IL-4) release in PMACI-treated HMC-1 cells. Values represent the mean values±S.E. of three independent experiments. *** represents that the p-value is less than 0.001 (p<0.001) compared with CON. # represents that the p-value is less than 0.05 (p<0.05) compared with EXP 1. ## represents that the p-value is less than 0.01 (p<0.01) compared with EXP1.

FIG. 6 provides a graph illustrating the inhibitory effects of Tβ4 at above about 1 μg/ml concentration on interleukin 13 (IL-13) release in PMACI-treated HMC-1 cells. Values represent the mean values±S.E. of three independent experiments. *** represents that the p-value is less than 0.001 (p<0.001) compared with CON. # represents that the p-value is less than 0.05 (p<0.05) compared with EXP 1. ## represents that the p-value is less than 0.01 (p<0.01) compared with EXP1.

FIG. 7 provides a graph illustrating the inhibitory effects of Tβ4 at above about 1 μg/ml concentration on tumor necrosis factor-alpha (TNF-α) release in PMACI-treated HMC-1 cells. Values represent the mean values±S.E. of three independent experiments. *** represents that the p-value is less than 0.001 (p<0.001) compared with CON. ## represents that the p-value is less than 0.01 (p<0.01) compared with EXP 1. ### represents that the p-value is less than 0.001 (p<0.001) compared with EXP 1.

### DETAILED DESCRIPTION

The detailed description of the disclosed subject matter is divided into the following subsections for clarity and not by way of limitation:

I. Definitions;

II. Pharmaceutical Compositions; and

III. Methods.

### I. Definitions

The terms used in this specification generally have their ordinary meanings in the art, within the context of the disclosed subject matter and in the specific context where each term is used. Certain terms are discussed below, or elsewhere in the specification, to provide additional guidance to the practitioner in describing the compositions and methods of the disclosed subject matter.

As used herein, the following terms have the meanings ascribed to them below, unless specified otherwise. Abbreviations used herein have their conventional meaning within the chemical and biological arts.

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example, within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about. About with respect to a concentration range of the compositions of the current disclosure also refers to any variation of a stated amount or range, which would be an effective amount or range.

As used herein, the term “additive” can mean any additional components that can be added to the composition as described herein. One or more additives can be added to the composition. Exemplary additives can include preservatives, viscosity agents, buffering agents, hypertonic agents, isotonic agents, and pH adjustment agents. Additives in the current disclosure can be used in any suitable amount.

As used herein, the term “administering” can mean any suitable route (e.g., via topical administration).

The term “agent,” as used herein, means a substance that produces or is capable of producing an effect and would include, but is not limited to, chemicals, pharmaceuticals, biologics, small organic molecules, antibodies, nucleic acids, peptides, and proteins.

As used herein, the term “co-administer” is meant that a composition described herein is administered at the same time, just prior to, or just after the administration of additional therapies. The composition of the disclosure can be administered alone or can be co-administered with a second composition and/or therapeutic agent to a subject. Co-administration is meant to include simultaneous or sequential administration of the composition individually or in combination with a second composition and/or therapeutic agent. Additionally, the first and second agents can be formulated separately or together in one or more compositions.

As used herein, “composition” refers to a composition intended for various applications to a target tissue (e.g., eye and skin) or its related or surrounding tissues. The term also includes compositions intended to therapeutically treat conditions of the target tissue itself or the tissues surrounding the target tissue. The composition can be applied topically or by other techniques, known to persons skilled in the art, such as injection. Examples of suitable topical administration include administration in solution drops, gels, creams, ointments, and spray formulations. A further suitable topical administration route is by subconjunctival and/or subdermal injection.

As used herein, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “concurrent administration” includes overlapping in duration at least in part. For example, when two agents (e.g., any of the compositions described herein) are administered concurrently, their administration occurs within a certain desired time. The compositions' administration can begin and end on the same day. The administration of one composition can also precede the administration of a second composition by day(s) as long as both compositions are taken on the same day at least once. Similarly, the administration of one composition can extend beyond the administration of a second composition as long as both compositions are taken on the same day at least once. The compositions do not have to be taken at the same time each day to include concurrent administration.

As used herein, “conservative variant” or grammatical variations thereof can denote the replacement of an amino acid residue by another biologically similar residue. Examples of conservative variations include the replacement of a hydrophobic residue, such as isoleucine, valine, leucine, or methionine for another, the replacement of a polar residue for another, such as the substitution of arginine for lysine, glutamic for aspartic acids, or glutamine for asparagine, and the like.

As used herein, the term “cream” can refer to a thick (high viscosity) liquid or semi-liquid that can be used for the therapeutic treatment of a disease, syndrome, or condition.

As used herein, the term “dispersion” can refer to a multi-phase liquid medium. For example, a first phase (e.g., particles) can be dispersed throughout a second phase, which can be a continuous phase. The dispersion can be formed when at least two non-mixable phases are compounded.

The term “dosage” is intended to encompass a formulation expressed in terms of total amounts for a given timeframe, for example, as μg/kg/hr, μg/kg/day, mg/kg/day, or mg/kg/hr. The dosage is the amount of an ingredient administered in accordance with a particular dosage regimen. A “dose” is an amount of an agent administered to a mammal in a unit volume or mass, e.g., an absolute unit dose expressed in mg of the agent. The dose depends on the concentration of the agent in the formulation, e.g., in moles per liter (M), mass per volume (m/v), or mass per mass (m/m). The two terms are closely related, as a particular dosage results from the regimen of administration of a dose or doses of the formulation. The particular meaning, in any case, will be apparent from the context.

As used herein, an “effective amount” or “therapeutically effective amount” is that amount sufficient to affect a desired biological effect, such as beneficial results, including clinical results. As such, an “effective amount” depends upon the context in which it is being applied. An effective amount can vary according to factors known in the art, such as the disease state, age, sex, and weight of the individual being treated. Several divided doses can be administered daily, or the dose can be proportionally reduced as indicated by the exigencies of the therapeutic situation. In addition, the compositions/formulations of this disclosure can be administered as frequently as necessary to achieve a therapeutic amount.

As used herein, the term “fragment” or “peptide” or “peptide fragment” comprises a portion of a protein (e.g., Tβ4 protein) with homology or percent amino acid sequence identity. Peptides can be biologically occurring short chains of amino acid monomers linked by peptide (amide) bonds.

As used herein, “gel” can refer to a material that is not a readily flowable liquid and is not a solid, i.e., a semi-solid gel. Gels can be formed from naturally occurring or synthetic materials. The gels can be non-ordered to slightly ordered, showing some birefringence, liquid crystal character. A semi-solid gel formulation viscosity can increase with concentration. Gels can be administered topically.

As used herein, “homology” or “percent (%) amino acid sequence identity” is used with respect to a protein (i.e., Tβ4 or fragment thereof). The homology or percent amino acid sequence identity can be defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to or can be applied to, the compliment of a test sequence. The definition also includes sequences that have deletions and/or additions, as well as those that have substitutions. Alignment for purposes of determining percent amino acid sequence identity can be achieved in various ways that are within the skill in the art, for instance, using publicly available computer software such as BLAST, BLAST-2, or ALIGN software. Those skilled in the art can determine appropriate parameters for measuring alignment, including any algorithms needed to achieve maximal alignment over the full length of the sequences being compared.

As used herein, “liquid” is a dosage form consisting of a composition in its liquid state. A liquid is pourable; it flows and conforms to its container at room temperature. Liquids display Newtonian or pseudoplastic flow behavior. In certain embodiments, a “semi-liquid” as used herein can have properties of both a liquid and another formulation (i.e., a suspension, an emulsion, a solution, a cream, a gel, a jelly, and the like).

As used herein, “ointment” can refer to a highly viscous liquid or semi-liquid formulation that can be used for the therapeutic treatment of a disease, syndrome, or condition.

As used herein, “patient,” “patient in need thereof,” “subject,” and “subject in need thereof” are used interchangeably and refer to an animal or living organism (human or nonhuman) suffering from or prone to a disease or condition that can be treated by administration using the methods and compositions provided herein. Non-limiting examples of subjects include humans, other mammals, bovines, rats, mice, dogs, monkeys, goats, sheep, cows, deer, and other non-mammalian animals. In certain embodiments, the subject is human.

As used herein, “pharmaceutically acceptable carrier” includes any and all solvents, dispersion media, coatings, antibacterial and antifungal agents, isotonic and absorption delaying agents, and the like that are physiologically compatible. The type of carrier can be selected based upon the intended route of administration. Pharmaceutically acceptable carriers include sterile aqueous solutions or dispersions and sterile powders for the extemporaneous preparation of sterile topical solutions or dispersion. The use of such media and agents for pharmaceutically active substances is well known in the art. Except insofar as any conventional media or agent is incompatible with the composition (e.g., Tβ4 or fragments thereof), use thereof in the topical compositions for the disclosure is contemplated.

The term “preservative” as used herein can include any agents included in a topical composition for the purpose of inhibiting the growth of microorganisms (e.g., bacteria, fungi, viruses, and protozoa) in the product, thereby helping to maintain sterility during use. Additionally, the term “anti-microbial agent” can be used herein to denote a specific active agent that provides the anti-microbial efficacy. Exemplary preservatives can include, for example, benzalkonium chloride, thimerosal, chlorobutanol, chlorhexidine, methyl paraben, propyl paraben, phenylethyl alcohol, edetate disodium sorbic acid, Onamer M Polyquat, cetyl bromide, cetyl pyridinium chloride, benzyl bromide, EDTA, phenylmercury nitrate, phenylmercury acetate, thimerosal, merthiolate, acetate and phenylmercury borate, polymyxin B sulphate, methyl and propyl parabens, quaternary ammonium chloride, sodium benzoate, sodium propionate, and sodium perborate, and other agents known to those skilled in the art, or a combination thereof.

As used herein, the terms “prevent,” “preventing,” or “prevention,” “prophylactic treatment,” and the like refer to reducing the probability of developing a disorder or condition in a subject who does not have but is at risk of or susceptible to developing a disorder or condition. The prevention can be complete (i.e., no detectable symptoms) or partial so that fewer symptoms are observed than would likely occur absent treatment. The terms further include a prophylactic benefit. For disease or condition to be prevented, the compositions can be administered to a patient at risk of developing a particular disease or to a patient reporting one or more of the physiological symptoms of a disease, even though a diagnosis of this disease cannot have been made.

As used herein, “sequential administration” includes that the administration of two agents (e.g., compositions described herein) occurs separately on the same day or does not occur on the same day (e.g., occurs on consecutive days).

As used herein, a “solution” is a clear, homogeneous liquid dosage form that contains one or more chemical substances (i.e., Tβ4 or fragments thereof) dissolved in a solvent or mixture of mutually miscible solvents. A solution is a liquid preparation that contains one or more dissolved chemical substances in a suitable solvent or mixture of mutually miscible solvents. Because molecules of a drug substance in solution are uniformly dispersed, the use of solutions as dosage forms generally provides assurance of uniform dosage upon administration and good accuracy when the solution is diluted or otherwise mixed. For example and not limitation, Tβ4 can be dissolved in a solution comprised of sodium chloride, potassium chloride, calcium chloride, magnesium chloride, sodium acetate, and sodium citrate, with a pH of approximately 7.0.

The term “solvent,” as used herein, refers to a liquid solvent, either aqueous or non-aqueous. The selection of the solvent depends notably on the solubility of the composition of said solvent and on the mode of administration. The aqueous solvent can consist solely of water, or can consist of water plus one or more miscible solvents, and can contain dissolved solutes such as sugars, buffers, salts, or other excipients. The more commonly used non-aqueous solvents are the short-chain organic alcohols, such as methanol, ethanol, propanol, short-chain ketones, such as acetone, and poly alcohols, such as glycerol.

“Suspension,” as used herein, is a liquid dosage form that contains solid particles dispersed in a liquid vehicle.

As used herein, “thymosin beta 4” or “Tβ4” refers to a human protein. Tβ4 encodes for an actin sequestering protein, which plays a role in the regulation of actin polymerization. The protein is also involved in cell proliferation, migration, and differentiation. The thymosin beta 4 peptide, if used after a heart attack, has been shown to potentially reactivate cardiac progenitor cells to repair damaged heart tissue. The safety of topical Tβ4 formulations has been demonstrated, both in dermal preparations and in a preservative-free formulation used in the eye. Based on its multifunctional activities during tissue regeneration, Tβ4 has the potential for clinical application in a wide range of pathological conditions, including ocular or dermal diseases. The NCBI Reference Sequence of human Tβ4 is available under accession number NP 066932.1.

The terms “treat,” “treating” or “treatment,” and other grammatical equivalents as used herein include alleviating, abating, ameliorating, or preventing a disease, condition, or symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder.

As described herein, any concentration range, percentage range, ratio range, or integer range is to be understood to include the value of any integer within the recited range and, when appropriate, fractions thereof (such as one-tenth and one-hundredth of an integer), unless otherwise indicated.

### II. Pharmaceutical Compositions

The present disclosure provides pharmaceutical compositions comprising a therapeutically effective amount of thymosin beta 4 (Tβ4), Tβ4 fragments, Tβ4 isoforms, Tβ4 derivatives, or variants thereof, wherein the therapeutically effective amount is present in an amount to decrease release of a pruritus-related factor or decrease release of an inflammatory cytokine from a target tissue.

In certain embodiments, Tβ4 can be human Tβ4. The human Tβ4 is a polypeptide composed of 43 amino acids having 4.9 kDa, which can be first isolated from the thymus and then identified from various tissues. This protein can upregulate the migration and proliferation of corneal epithelial cells, reduce inflammation, increase stem cell recruitment, promote tissue healing and regeneration, and increase cell survival. In some embodiments, the pharmaceutical composition can include Tβ4 isoforms. Tβ4 isoforms can have about 70%, or about 75%, or about 80% or more homology to the known amino acid sequence of Tβ4. Such isoforms can include, for example, Tβ4^{ala}, Tβ9, Tβ10, Tβ11, Tβ12, Tβ13, Tβ14, and Tβ15. Tβ4 of the presently disclosed subject matter can also be an N-terminal variant or C-terminal variant of wild-type Tβ4.

For example and not limitation, other agents or proteins having anti-inflammatory activity and/or actin sequestering or binding capability, or that can mobilize actin or modulate actin polymerization, as demonstrated in an appropriate sequestering, binding, mobilization, or polymerization assay, or identified by the presence of an amino acid sequence that mediates actin binding, such as LKKTET [SEQ ID NO:1] or LKKTNT [SEQ ID NO:2], for example, can similarly be employed in the disclosed subject matter. Such proteins can include but are not limited to gelsolin, vitamin D binding protein (DBP), profilin, cofilin, DNaseI, villin, fragmin, severin, capping protein, β-actinin, and acumentin.

In certain embodiments, the pharmaceutical composition can include oxidized forms of Tβ4 including Tβ4 sulfoxide or conservative variant thereof. Oxidized Tβ4 is a form of Tβ4 in which a methionine residue, 6 amino acids from the N-terminus (Meth), is oxidized such that the residue is converted to methionine sulfoxide. The oxidized Tβ4 can be obtained by reacting native Tβ4 under oxidizing conditions, for example, by treating with hydrogen peroxide.

Although the disclosed subject matter is described primarily hereinafter with respect to Tβ4 and Tβ4 fragments, it is to be understood that the following description is intended to be equally applicable to conservative variants thereof and/or Tβ4 isoforms, analogues or derivatives, including oxidized Tβ4, N-terminal variants of Tβ4, and C-terminal variants of Tβ4.

In non-limiting embodiments, the therapeutically effective amount of Tβ4, Tβ4 fragments, Tβ4 isoforms, Tβ4 derivatives, or variants thereof in the pharmaceutical composition can be at least about 1 μg/ml, at least about 1.5 μg/ml, at least about 3 μg/ml, at least about 6 μg/ml, at least about 10 μg/ml, at least about 50 μg/ml, at least about 100 μg/ml, at least about 200 μg/ml, at least about 300 μg/ml, at least about 500 μg/ml, at least about 1 mg/ml, at least about 5 mg/ml, at least about 10 mg/ml, at least about 15 mg/ml, or at least about 20 mg/ml. In non-limiting embodiments, the effective amount can be from about 1 μg/ml to about 10 mg/ml, from about 1 μg/ml to about 1 mg/ml, from about 1 μg/ml to about 500 μg/ml, from about 1 μg/ml to about 300 μg/ml, from about 1 μg/ml to about 100 μg/ml, from about 1 μg/ml to about 10 μg/ml, from about 10 μg/ml to about 10 mg/ml, from about 10 μg/ml to about 5 mg/ml, from about 10 μg/ml to about 1 mg/ml, from about 10 μg/ml to about 500 μg/ml, from about 10 μg/ml to about 300 μg/ml, from about 10 μg/ml to about 100 μg/ml, from about 50 μg/ml to about 10 mg/ml, from about 50 μg/ml to about 5 mg/ml, from about 50 μg/ml to about 1 mg/ml, from about 50 μg/ml to about 500 μg/ml, from about 50 μg/ml to about 300 μg/ml, from about 100 μg/ml to about 10 mg/ml, from about 100 μg/ml to about 5 mg/ml, from about 100 μg/ml to about 3 mg/ml, from about 100 μg/ml to about 2 mg/ml, from about 100 μg/ml to about 1 mg/ml, from about 100 μg/ml to about 500 μg/ml, or from about 100 μg/ml to about 300 μg/ml.

The pharmaceutical composition containing the active compound (e.g., Tβ4) can contain a physiologically compatible vehicle, as those skilled in the art can select using conventional criteria. In non-limiting embodiments, the pharmaceutical composition can be formulated for topical administration, systemic administration, local injection to target tissues, or as an oral spray, nasal spray, inhaler, or mouth wash. For example, the pharmaceutical composition can be in the form of a solution, lotion, plaster, gel, cream, paste, spray, suspension, dispersion, hydrogel, ointment, oil, patch, lens, nasal spray, or foaming agent.

In certain embodiments, the pharmaceutical composition can include a pharmaceutically acceptable carrier. For example, the pharmaceutical composition can include a carrier that can be suitable for topical administration. The carriers can include, for example and not limitation, water; a mixture of water and water-miscible solvents such as C₁-C₇ alkanols, vegetable oils, or mineral oils such as from about 0.5 to about 5 wt. % of hydroxyethyl cellulose, ethyl oleate, carboxymethyl cellulose, polyvinyl pyrrolidone, and other non-toxic water-soluble polymers, for example, cellulose derivatives such as methyl cellulose, alkali-metal salts of carboxymethyl cellulose, hydroxymethyl cellulose, hydroxyethyl cellulose, hydroxypropyl methyl cellulose, and hydroxypropyl cellulose, acrylates or methacrylates such as salts of polyacrylate or ethyl acrylate, polyacrylamides; natural products such as beeswax, gelatin, alginate, pectin, tragacanth, karaya gum, xanthan gum, gellan gum, hyaluronic acid, and its pharmaceutically acceptable salts, carrageenan, agar, acacia, starch derivatives such as starch acetate and hydroxylpropyl starch; and other synthetic products, for example, carbomer, polyvinyl alcohol, polyvinyl pyrrolidone, polyvinyl methylether, polyethylene oxide, preferably, cross-linked polyacrylic acid such as neutral carbopol, or mixtures of the above polymers. Preferable carriers can include water, cellulose derivatives, for example, methyl cellulose, alkali-metal salts of carboxymethyl cellulose, hydroxymethyl cellulose, hydroxyethyl cellulose, hydroxypropyl methyl cellulose and hydroxypropyl cellulose, neutral carbopol, or mixtures thereof.

In certain embodiments, the pharmaceutical composition can include one or more pharmaceutically acceptable excipients including but not limited to stabilizers, buffers, preservatives, tonicity agents, slow-release agents, and viscosity enhancers. In non-limiting embodiments, the amount and type of excipient(s) added can be varied depending on specific requirements. The excipient(s) can be used in a range of about 0.0001 to about 90 wt. %, and within the range commonly used in the art.

In certain embodiments, the composition can include stabilizers. The stabilizers can include, for example, and not limitation, tyloxapol, aliphatic glycerol poly-lower alkylene glycol esters, aliphatic poly-lower alkylene glycol esters, polyethylene glycols, glycerol ethers, acetic acid, citric acid, ascorbic acid, EDTA/disodium edetate, glutathione, acetylcysteine or mixtures of these compounds. Acetic acid used herein is a weak acid represented by the formula CH₃COOH. In the presently disclosed subject matter, the stabilizers can be used in the form of acetate. The acetate can include at least one molecule of water. For example and not limitation, mono-, sesqui-, di-, tri-, tetra-, penta-, hexa-, hepta-, octa-, nona-, deca-, undeca-, or dodeca-hydrate forms of acetate can be added into the composition. In some embodiments, sodium acetate trihydrate can be included in an amount of from about 0.01% (w/v) to about 1.5% (w/v) based on the total volume of the composition. In non-limiting embodiments, acetic acid or its salt can be included in an amount of from about 0.1% (w/v) to about 0.8% (w/v), and from about 0.2% (w/v) to about 0.5% (w/v). Citric acid used herein can be a compound represented by formula C₆H₈O₇. In some embodiments, citric acid can be used in the form of one or more citrates. Citrate can be a derivative of citric acid. In non-limiting embodiments, the citrate can include at least one molecule of water. For example and not limitation, mono-, sesqui-, di-, tri-, tetra-, penta-, hexa-, hepta-, octa-, nona-, deca-, undeca-, or dodeca-hydrate forms of citrate can be added into the composition. In some embodiments, the citrate can be sodium citrate and sodium citrate dihydrate. In these forms, citric acid or its salt can be included in an amount of from about 0.01% (w/v) to about 0.5% (w/v). Further, citric acid or its salt can be included in an amount of from about 0.05% (w/v) to about 0.25% (w/v), and from about 0.1% (w/v) to about 0.3% (w/v). They can be added in an amount sufficient to dissolve active ingredients.

In certain embodiments, the composition can include a buffer. For example, the buffer can include any forms of acetate, ascorbate, borate, hydrocarbonate/carbonate, gluconate, phosphate, propionate, acetic acid, citric acid, and/or tromethamine (TRIS) buffers. The buffer can be added, for example, in an amount to ensure and maintain a physiologically acceptable pH range. Such pH can be in the range of from about 3.5 to about 9, from about 4 to about 7, from about 4.5 to about 6.5, or from about 5 to about 6, from about 5 to about 6, from about 5.5 to about 6.0, from about 6 to about 8.2, or from about 6.8 to about 8.1.

In certain embodiments, the pH value of the formulations can range from about 3.5 to about 9, from about 3.5 to about 7, from about 4.5 to about 6.5, or from about 5 to about 6, from about 5 to about 6, from about 5.5 to about 6.0, from about 4.5 to about 8, and from about 5.5 to about 7.8, and can be about pH 7.0.

In certain embodiments, the composition in accordance with the presently disclosed subject matter can further include an acid selected from the group consisting of hydrochloric acid, acetic acid, phosphoric acid, or a combination thereof. In non-limiting embodiments, the composition can further include a base selected from the group consisting of sodium hydroxide, potassium hydroxide, sodium hydrogen carbonate, and a combination thereof. For example and not limitation, hydrochloric acid or sodium hydroxide can be suitably added to adjust the pH of the composition. As such, the pH of the composition can be from about 3.5 to about 9, from about 4 to about 7, from about 4.5 to about 6.5, or from about 5 to about 6, from about 5 to about 6, from about 5.5 to about 6.0, from about 6 to about 8.2, or from about 6.8 to about 8.1.

In certain embodiments, the composition can include preservatives. The preservatives can include, for example, quaternary ammonium salts such as Cetrimide, benzalkonium chloride, benzoxonium chloride, alkyl-mercury salts of thiosalicylic acid such as thimerosal, phenylmercuric nitrate, phenylmercuric acetate or phenylmercuric borate, parabens such as phenylparaben or propylparaben, alcohols such as chlorobutanol, benzyl alcohol or phenyl ethanol, guanidine derivatives such as chlorohexidine or polyhexamethylene biguanide or sorbic acid. The preservative can be added in a sufficient amount to prevent secondary contamination caused by bacteria and fungi during the use.

In certain embodiments, the composition can include a tonicity agent to adjust the composition closer to isotonicity (e.g., 0.9% saline). For example and not limitation, any form of sodium chloride, potassium chloride, calcium chloride, magnesium chloride, dextrose, mannitol, or combinations thereof can be added to the composition comprising Tβ4. In non-limiting embodiments, the tonicity agents can include at least one molecule of water. For example and not limitation, mono-, sesqui-, di-, tri-, tetra-, penta-, hexa-, hepta-, octa-, nona-, deca-, undeca-, or dodeca-hydrate forms of sodium chloride, potassium chloride, calcium chloride, magnesium chloride, or combinations thereof can be added into the composition. An amount of the tonicity agent depends upon the kind of active agents to be added. For example, the composition of the present disclosed subject matter can include a tonicity agent therein to enable the final composition to have an osmolality acceptable for appropriate use (e.g., a range of from about 150 to about 450 mOsm or from about 250 to about 350 mOsm). The tonicity agents can include, for example, sodium salts, potassium salts, sodium chloride, and/or potassium chloride. In non-limiting embodiments, a concentration of sodium chloride can range from about 0.1 to about 1.2% (w/v), from about 0.3 to about 1.0% (w/v), or from about 0.5 to about 0.7% (w/v). A concentration of potassium chloride can range from about 0.01 to about 0.15% (w/v), from about 0.03 to about 0.12% (w/v), or from about 0.05 to about 0.09% (w/v). A concentration of calcium chloride dihydrate can range from about 0.01 to about 0.12% (w/v), from about 0.03 to about 0.09% (w/v), or from about 0.03 to about 0.06% (w/v). A concentration of magnesium chloride hexahydrate can range from about 0.01 to about 0.12% (w/v) or from about 0.01 to about 0.05% (w/v). Although the tonicity agents are described primarily herein with respect to adjusting the tonicity of the composition, the disclosed tonicity agents can also be used as electrolytes.

In certain embodiments, the composition can include a viscosity enhancer. The viscosity enhancers can include but are not limited to (a) Monomeric polyols, such as tyloxapol (from about 0.1 to about 1%), glycerol (from about 0.2 to about 1%), propylene glycol (from about 0.2 to about 1%), ethylene glycol (from about 0.2 to about 1%); (b) Polymeric polyols, such as polyethylene glycol (e.g., PEG 300, PEG 400) (from about 0.2 to about 1%); (c) Cellulose derivatives (polymers of the cellulose family), such as hydroxyethylcellulose (from about 0.2 to about 2.5%), hypromellose (from about 0.2 to about 2.5%), hydroxypropylmethyl cellulose (from about 0.2 to about 2.5%), methylcellulose (from about 0.2 to about 2.5%), carboxymethylcellulose sodium (from about 0.2 to about 2.5%), hydroxylpropylcellulose (from about 0.2 to about 2.5%); (d) Dextrans, such as dextran 70 (at about 0.1% when used with another polymeric demulcent agent); (e) Water-soluble proteins such as gelatin (at about 0.01%); (f) Vinyl polymers such as polyvinyl alcohol (from about 0.1 to about 4%), polyvinyl pyrrolidine (from about 0.1 to about 4%); (g) Other polyols, such as polysorbate 80 (from about 0.2 to about 1%), povidone (from about 0.1 to about 2%); (h) Carbomers, such as carbomer 934P, carbomer 941, carbomer 940, and carbomer 974P, and (i) Polysaccharides/Glycosaminoglycans, such as hyaluronan (hyaluronic acid/hyaluronate) (from about 0.1 to about 3%), chondroitin sulfate (from about 0.1 to about 3%).

In certain embodiments, the pharmaceutical composition can be prepared for intravenous, intraperitoneal, intramuscular, or subcutaneous injections, or inhalation (nasally), transdermal, suppository, or enema administration. In non-limiting embodiments, the pharmaceutical compositions of the present disclosure can be formulated using pharmaceutically acceptable carriers well known in the art that are suitable for parenteral administration. The terms “parenteral administration” and “administered parenterally,” as used herein, refers to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, and intrasternal injection and infusion. For example, and not by way of limitation, pharmaceutical compositions of the present disclosure can be administered to the patient intravenously in a pharmaceutically acceptable carrier such as physiological saline. In certain embodiments, the present disclosure provides a parenteral pharmaceutical composition comprising compositions disclosed herein.

In non-limiting embodiments, the disclosed composition can include a therapeutically effective amount of Tβ4, Tβ4 fragments, Tβ4 isoforms, Tβ4 derivatives, or variants thereof, wherein the therapeutically effective amount can be present in an amount to decrease the release of a pruritus-related factor or decrease the release of an inflammatory cytokine from a target tissue, one or more pharmaceutically acceptable excipients, and a pharmaceutically acceptable carrier. In non-limiting embodiments, the therapeutically effective amount of Tβ4, Tβ4 fragments, Tβ4 isoforms, Tβ4 derivatives, or variants thereof in the pharmaceutical composition can be from about 50 μg/ml to about 10 mg/ml or from about 100 μg/ml to about 1 mg/ml. The pharmaceutically acceptable excipients can include stabilizers, buffers, preservatives, tonicity agents, slow-release agents, viscosity enhancers, or combinations thereof. In non-limiting embodiments, the pharmaceutically acceptable carrier can include water, cellulose derivatives, methyl cellulose, alkali-metal salts of carboxymethyl cellulose, hydroxymethyl cellulose, hydroxyethyl cellulose, hydroxypropyl methyl cellulose, hydroxypropyl cellulose, neutral carbopol, or mixtures thereof. In non-limiting embodiments, the composition can be in a form of a solution, lotion, plaster, gel, cream, paste, spray, suspension, dispersion, hydrogel, ointment, oil, patch, lens, inhaler, nasal spray, or foaming agent. In non-limiting embodiments, the pruritus-related factor can include histamine, leukotriene β4, prostaglandin E2, interleukin 4, interleukin 13, tumor necrosis factor α, and combinations thereof.

In non-limiting embodiments, the disclosed composition can include a therapeutically effective amount of Tβ4, Tβ4 fragments, Tβ4 isoforms, Tβ4 derivatives, or variants thereof, wherein the therapeutically effective amount can be present in an amount to decrease the release of a pruritus-related factor or decrease the release of an inflammatory cytokine from a target tissue, wherein the therapeutically effective amount of Tβ4, Tβ4 fragments, Tβ4 isoforms, Tβ4 derivatives, or variants thereof in the pharmaceutical composition can be from about 50 μg/ml to about 10 mg/ml, from about 50 μg/ml to about 5 mg/ml, from about 50 μg/ml to about 1 mg/ml, or alternately, from about 100 μg/ml to about 1 mg/ml, wherein the pruritus-related factor can be selected from the group consisting of an allergic reaction factor, an inflammatory cytokine, and a combination thereof, wherein allergic reaction factor can be selected from the group consisting of histamine, leukotriene β4, prostaglandin E2, and combinations thereof, wherein the inflammatory cytokine can be selected from the group consisting of interleukin 4, interleukin 13, tumor necrosis factor α, and combinations thereof, and wherein the composition can be in the form of a solution, lotion, plaster, gel, cream, paste, spray, suspension, dispersion, hydrogel, ointment, oil, patch, lens, inhaler, nasal spray, or foaming agent.

### III. Methods

The present disclosure provides a method of treating and/or preventing pruritus. The method includes administering a therapeutically effective amount of Tβ4, Tβ4 fragments, Tβ4 isoforms, Tβ4 derivatives, or variants thereof, wherein the therapeutically effective amount can decrease release of a pruritus-related factor or decrease release of an inflammatory cytokine from a target tissue.

Pruritus refers to itching, which can be accompanied by an unpleasant sensation that can cause the desire to scratch or rub the tissue (e.g., skin, eye, and nose). Pruritus can be caused by contact with toxic animals and plants, psoriasis, seborrheic dermatitis, or fungal growth. Pruritus can also be caused by allergic reactions to non-toxic substances that are not toxic substances.

Pruritus can include various signs and symptoms including, but not limited to, redness, bumps, blisters, cracked skin, scaly patches, or combinations thereof. A subject with pruritus can exhibit one or more signs or symptoms. The presently disclosed subject matter provides methods for treating or preventing the signs and symptoms of pruritus in a subject by reducing the release of a pruritus-related factor or an inflammatory cytokine from target tissue via administration of a therapeutically effective amount of Tβ4, Tβ4 fragments, Tβ4 isoforms, Tβ4 derivatives, or variants thereof.

The present disclosure also provides a method of treating pruritus. The method of treating pruritus includes administering a composition that comprises a therapeutically effective amount of Tβ4, Tβ4 fragments, Tβ4 isoforms, Tβ4 derivatives, or variants thereof, wherein the therapeutically effective amount can decrease release of a pruritus-related factor or decrease release of an inflammatory cytokine from target tissue to a subject.

In certain embodiments, the pruritus can be treated by alleviating, abating, or reducing the pruritus-related factor. In non-limiting embodiments, the pruritus-related factor can be an allergic reaction factor that can be secreted from target cells or tissues to cause allergic reactions. In non-limiting embodiments, the allergic reaction factor can be histamine, leukotriene β4 (LTB4), prostaglandin E2 (PGE2), or combinations thereof.

In certain embodiments, administration of the disclosed subject matter for treating pruritus can reduce the release of histamine from target cells or tissues. In non-limiting embodiments, the release of the histamine can be reduced by at least about 1%, at least about 3%, at least about 5%, at least about 10%, at least about 15%, at least about 20%, at least about 25%, at least about 30%, at least about 35%, or at least about 40%. For example, the release of histamine from mast cells can be reduced by at least about 20% after delivering the pharmaceutical composition to the mast cells.

In certain embodiments, administration of the disclosed subject matter for treating pruritus can reduce the release of LTB4 from target cells or tissues. In non-limiting embodiments, the release of the LTB4 can be reduced by at least about 1%, at least about 3%, at least about 5%, at least about 10%, at least about 15%, at least about 20%, at least about 25%, at least about 30%, at least about 35%, or at least about 40%. For example, the release of LTB4 from mast cells can be reduced by at least about 10% after delivering the pharmaceutical composition to the mast cells.

In certain embodiments, administration of the disclosed subject matter for treating pruritus can reduce the release of PGE2 from target cells or tissues. In non-limiting embodiments, the release of the PGE2 can be reduced by at least about 1%, at least about 3%, at least about 5%, at least about 10%, at least about 15%, at least about 20%, at least about 25%, at least about 30%, at least about 35%, or at least about 40%. For example, the release of PGE2 from mast cells can be reduced by at least about 10% after delivering the pharmaceutical composition to the mast cells.

In certain embodiments, the release of inflammatory cytokines from a target tissue can be reduced for treating pruritus. In non-limiting embodiments, the pruritus-related factor can be an inflammatory cytokine that can be secreted from target cells or tissues to cause allergic inflammation. In non-limiting embodiments, the inflammatory cytokine can be interleukin-4 (IL-4), interleukin-13 (IL-13), Tumor necrosis factor-alpha (TNF-α), or combinations thereof.

In certain embodiments, administration of the disclosed subject matter for treating pruritus can reduce the release of IL-4 from target cells or tissues. In non-limiting embodiments, the release of the IL-4 can be reduced by at least about 10%, at least about 15%, at least about 20%, at least about 25%, at least about 30%, at least about 35%, or at least about 40%. For example, the release of IL-4 from mast cells can be reduced by at least about 20% after delivering the pharmaceutical composition to the mast cells.

In certain embodiments, administration of the disclosed subject matter for treating pruritus can reduce the release of IL-13 from target cells or tissues. In non-limiting embodiments, the release of the IL-13 can be reduced by at least about 10%, at least about 15%, at least about 20%, at least about 25%, at least about 30%, at least about 35%, or at least about 40%. For example, the release of IL-13 from mast cells can be reduced by at least about 15% after delivering the pharmaceutical composition to the mast cells.

In certain embodiments, administration of the disclosed subject matter for treating pruritus can reduce the release of TNF-α from target cells or tissues. In non-limiting embodiments, the release of the TNF-α can be reduced by at least about 10%, at least about 15%, at least about 20%, at least about 25%, at least about 30%, at least about 35%, or at least about 40%. For example, the release of TNF-α from mast cells can be reduced by at least about 25% after delivering the pharmaceutical composition to the mast cells.

In certain embodiments, the target tissue can be skin tissue, connective tissue, nerve tissue, soft tissue, epithelial tissue (e.g., oral, pharyngeal, nasal, ocular, and gastric), or combinations thereof for treating pruritus. In non-limiting embodiments, the target cell can be a mast cell in any tissues or keratinocytes.

The present disclosure also provides a method of preventing pruritus. The method includes administering a composition that comprises a therapeutically effective amount of Tβ4, Tβ4 fragments, Tβ4 isoforms, Tβ4 derivatives, or variants thereof, wherein the therapeutically effective amount can decrease release of a pruritus-related factor or decrease release of an inflammatory cytokine from the target tissue to a subject. For example and not limitation, the probability of developing signs and symptoms of pruritus can be reduced by administering the disclosed composition to a subject who does not have but is at risk of or susceptible to developing the pruritus conditions.

In certain embodiments, the probability of developing signs and symptoms of pruritus can be reduced by abating or reducing the pruritus-related factor. In non-limiting embodiments, the pruritus-related factor can be an allergic reaction factor that can be secreted from target cells or tissues to cause allergic reactions. In non-limiting embodiments, the allergic reaction factor can be histamine, leukotriene β4 (LTB4), prostaglandin E2 (PGE2), or combinations thereof.

In certain embodiments, administration of the disclosed subject matter for preventing pruritus can reduce the release of histamine from target cells or tissues. In non-limiting embodiments, the release of the histamine can be reduced by at least about 1%, at least about 3%, at least about 5%, at least about 10%, at least about 15%, at least about 20%, at least about 25%, at least about 30%, at least about 35%, or at least about 40%. For example, the release of histamine from mast cells can be reduced by at least about 20% after delivering the pharmaceutical composition to the mast cells.

In certain embodiments, administration of the disclosed subject matter for preventing pruritus can reduce the release of LTB4 from target cells or tissues. In non-limiting embodiments, the release of the LTB4 can be reduced by at least about 1%, at least about 3%, at least about 5%, at least about 10%, at least about 15%, at least about 20%, at least about 25%, at least about 30%, at least about 35%, or at least about 40%. For example, the release of LTB4 from mast cells can be reduced by at least about 10% after delivering the pharmaceutical composition to the mast cells.

In certain embodiments, administration of the disclosed subject matter for preventing pruritus can reduce the release of PGE2 from target cells or tissues. In non-limiting embodiments, the release of the PGE2 can be reduced by at least about 1%, at least about 3%, at least about 5%, at least about 10%, at least about 15%, at least about 20%, at least about 25%, at least about 30%, at least about 35%, or at least about 40%. For example, the release of PGE2 from mast cells can be reduced by at least about 10% after delivering the pharmaceutical composition to the mast cells.

In certain embodiments, the release of inflammatory cytokines from a target tissue can be reduced to prevent pruritus. In non-limiting embodiments, the pruritus-related factor can be an inflammatory cytokine that can be secreted from target cells or tissues to cause allergic inflammation. In non-limiting embodiments, the inflammatory cytokine can be interleukin-4 (IL-4), interleukin-13 (IL-13), Tumor necrosis factor-alpha (TNF-α), or combinations thereof.

In certain embodiments, administration of the disclosed subject matter for preventing pruritus can reduce the release of IL-4 from target cells or tissues. In non-limiting embodiments, the release of the IL-4 can be reduced by at least about 10%, at least about 15%, at least about 20%, at least about 25%, at least about 30%, at least about 35%, or at least about 40%. For example, the release of IL-4 from mast cells can be reduced by at least about 20% after delivering the pharmaceutical composition to the mast cells.

In certain embodiments, administration of the disclosed subject matter for preventing pruritus can reduce the release of IL-13 from target cells or tissues. In non-limiting embodiments, the release of the IL-13 can be reduced by at least about 10%, at least about 15%, at least about 20%, at least about 25%, at least about 30%, at least about 35%, or at least about 40%. For example, the release of IL-13 from mast cells can be reduced by at least about 15% after delivering the pharmaceutical composition to the mast cells.

In certain embodiments, administration of the disclosed subject matter for preventing pruritus can reduce the release of TNF-α from target cells or tissues. In non-limiting embodiments, the release of the TNF-α can be reduced by at least about 10%, at least about 15%, at least about 20%, at least about 25%, at least about 30%, at least about 35%, or at least about 40%. For example, the release of TNF-α from mast cells can be reduced by at least about 25% after delivering the pharmaceutical composition to the mast cells.

In certain embodiments, the target tissue can be skin tissue, connective tissue, nerve tissue, soft tissue, epithelial tissue (e.g., oral, pharyngeal, nasal, ocular, and gastric), or combinations thereof for preventing pruritus. In non-limiting embodiments, the target cell can be a mast cell in any tissues or keratinocytes.

Dosage Regimens

For example and not limitation, the administration of the disclosed subject matter can be topical or systemic administration. An example of topical administration can include direct application of the composition in the form of a solution, lotion, plaster, gel, cream, paste, spray, suspension, dispersion, hydrogel, ointment, oil, patch, lens, inhaler, nasal spray, or foaming agent to a subject in order to contact the disclosed composition with target tissue and/or cells. An example of systemic administration includes intravenous, intraperitoneal, intramuscular, or subcutaneous injections, or inhalation (orally or nasally), transdermal, suppository, or enema administration of a composition containing Tβ4, Tβ4 fragments, Tβ4 isoforms, Tβ4 derivatives, or variants thereof as an active ingredient.

In certain embodiments, the following dosage regimens can be used to treat and/or prevent pruritus and can be used to reduce the release of the allergic reaction factor and the inflammatory cytokine from the target tissue and/or cell. In some embodiments, the composition for topical application can include the Tβ4, Tβ4 fragments, Tβ4 isoforms, Tβ4 derivatives, or variants thereof at least about 1 μg/ml, at least about 1.5 μg/ml, at least about 3 μg/ml, at least about 6 μg/ml, at least about 10 μg/ml, at least about 50 μg/ml, at least about 100 μg/ml, at least about 200 μg/ml, at least about 300 μg/ml, at least about 500 μg/ml, at least about 1 mg/ml, at least about 5 mg/ml, at least about 10 mg/ml, at least about 15 mg/ml, or at least about 20 mg/ml. In non-limiting embodiments, the effective amount of the Tβ4, Tβ4 fragments, Tβ4 isoforms, Tβ4 derivatives can be from about 1 μg/ml to about 10 mg/ml, from about 1 μg/ml to about 1 mg/ml, from about 1 μg/ml to about 500 μg/ml, from about 1 μg/ml to about 300 μg/ml, from about 1 μg/ml to about 100 μg/ml, from about 1 μg/ml to about 10 μg/ml, from about 10 μg/ml to about 10 mg/ml, from about 10 μg/ml to about 5 mg/ml, from about 10 μg/ml to about 1 mg/ml, from about 10 μg/ml to about 500 μg/ml, from about 10 μg/ml to about 300 μg/ml, from about 10 μg/ml to about 100 μg/ml, from about 50 μg/ml to about 10 mg/ml, from about 50 μg/ml to about 5 mg/ml, from about 50 μg/ml to about 1 mg/ml, from about 50 μg/ml to about 500 μg/ml, from about 50 μg/ml to about 300 μg/ml, from about 100 μg/ml to about 10 mg/ml, from about 100 μg/ml to about 5 mg/ml, from about 100 μg/ml to about 3 mg/ml, from about 100 μg/ml to about 2 mg/ml, from about 100 μg/ml to about 1 mg/ml, from about 100 μg/ml to about 500 μg/ml, or from about 100 μg/ml to about 300 μg/ml.

In certain embodiments, the composition for topical application can be administered to the target tissue and/or cell for treating and/or preventing pruritus by applying the composition in the form of a solution, lotion, plaster, gel, cream, paste, spray, suspension, dispersion, hydrogel, ointment, oil, patch, lens, inhaler, nasal spray, or foaming agent to the target region 1, 2, 3, 4, 8, 12, 18, or 24 times a day, or more. In non-limiting embodiments, the pharmaceutical composition can be applied once daily or twice daily, or three times daily, or four times daily, or five times daily, or 6 times daily.

In certain embodiments, the composition for the systemic application can include the Tβ4, Tβ4 fragments, Tβ4 isoforms, Tβ4 derivatives, or variants thereof at least about 1 μg/ml, at least about 1.5 μg/ml, at least about 3 μg/ml, at least about 6 μg/ml, at least about 10 μg/ml, at least about 50 μg/ml, at least about 100 μg/ml, at least about 200 μg/ml, at least about 300 μg/ml, at least about 500 μg/ml, at least about 1 mg/ml, at least about 5 mg/ml, at least about 10 mg/ml, at least about 15 mg/ml, or at least about 20 mg/ml. In non-limiting embodiments, the effective amount of the Tβ4, Tβ4 fragments, Tβ4 isoforms, Tβ4 derivatives can be from about 1 μg/ml to about 10 mg/ml, from about 1 μg/ml to about 1 mg/ml, from about 1 μg/ml to about 500 μg/ml, from about 1 μg/ml to about 300 μg/ml, from about 1 μg/ml to about 100 μg/ml, from about 1 μg/ml to about 10 μg/ml, from about 10 μg/ml to about 10 mg/ml, from about 10 μg/ml to about 5 mg/ml, from about 10 μg/ml to about 1 mg/ml, from about 10 μg/ml to about 500 μg/ml, from about 10 μg/ml to about 300 μg/ml, from about 10 μg/ml to about 100 μg/ml, from about 50 μg/ml to about 10 mg/ml, from about 50 μg/ml to about 5 mg/ml, from about 50 μg/ml to about 1 mg/ml, from about 50 μg/ml to about 500 μg/ml, from about 50 μg/ml to about 300 μg/ml, from about 100 μg/ml to about 10 mg/ml, from about 100 μg/ml to about 5 mg/ml, from about 100 μg/ml to about 3 mg/ml, from about 100 μg/ml to about 2 mg/ml, from about 100 μg/ml to about 1 mg/ml, from about 100 μg/ml to about 500 μg/ml, or from about 100 μg/ml to about 300 μg/ml.

In certain embodiments, the composition for the systemic application can be prepared in the liquid solutions or suspensions in aqueous physiological buffer solutions for parenteral administration or in the form of powders, drops, or aerosols for intranasal, intrapharyngeal, or ocular administration. The composition for the systemic application can be administered to the target tissue and/or cell for treating and/or preventing pruritus by delivering the composition prepared for systemic application to the target region 1, 2, 3, 4, 8, 12, 18, or 24 times a day, or more. In non-limiting embodiments, the pharmaceutical composition can be delivered once daily or twice daily, or three times daily, or four times daily, or five times daily, or 6 times daily.

In certain embodiments, the dosage administered can vary depending upon known factors, such as the route of administration, age, health, and weight of the recipient; nature and extent of symptoms, kind of concurrent treatment, frequency of treatment, and the effect desired. In non-limiting embodiments, the disclosed dosage regimes can be adjusted over time according to the individual need and the professional judgment of the person administering or supervising the administration of the composition. For example, the dosage of the composition can be increased if the lower dose does not provide sufficient activity in the treatment of the condition described herein (e.g., pruritus). Alternatively, the dosage of the composition can be decreased if the disease (e.g., pruritus) is reduced, no longer detectable, or eliminated.

In certain embodiments, the disclosed composition can be administered to the subject in a single dose or divided doses.

In certain embodiments, the duration of the disclosed treatment can be between about one day to about two years. In certain embodiments, the duration of the disclosed treatment can be about at least 1 day, at least 3 days, at least about 1 week, at least about 2 weeks, at least about 3 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, or at least about 24 months. In certain embodiments, the composition can be administered until the symptoms of pruritus are no longer detectable.

In certain embodiments, the disclosed compositions can be cyclically administered to a subject. Cycling therapy involves the administration of the compositions for a period of time, followed by a rest for a period of time, and repeating this sequential administration. Cycling therapy can reduce the development of resistance to one or more of the therapies, avoid or reduce the side effects of one of the therapies, and/or improves the efficacy of the treatment. In certain embodiments, the treatment stops after one cycle because the subject is intolerable to the adverse effects and toxicities associated with the disclosed compositions. In certain embodiments, the number of cycles can be more than about one cycle, about 2 cycles, about 3 cycles, about 4 cycles, about 5 cycles, about 6 cycles, about 7 cycles, about 8 cycles, about 9 cycles, about 10 cycles, about 11 cycles, about 12 cycles, about 13 cycles, about 14 cycles, or about 15 cycles.

Co-Administration

In certain embodiments, the composition disclosed herein can be used alone or in combination with one or more agents, e.g., anti-allergic agents. For example, but not by way of limitation, methods of the present disclosure can include administering the disclosed compositions and one or more anti-allergic agents. “In combination with,” as used herein, means that the disclosed composition, and the one or more agents, e.g., anti-allergic agents, can be administered to a subject as part of a treatment regimen or plan. In certain embodiments, being used in combination does not require that the composition and one or more agents, e.g., anti-allergic agents, are physically combined prior to administration, administered by the same route or that they be administered over the same time frame. In certain embodiments, the agent, e.g., anti-allergic agent, can be administered before the disclosed composition. In certain embodiments, the agent, e.g., anti-allergic agent, can be administered after the disclosed composition. In certain embodiments, the agent, e.g., anti-allergic agent, can be administered simultaneously with the disclosed composition. Non-limiting exemplary anti-allergic agents include, but are not limited to, anti-histamine agents, decongestants, steroids, mast cell stabilizers, leukotriene modifiers, nasal anticholinergics, epinephrine, and/or immunomodulator.

In certain embodiments, administering the disclosed compositions in combination with one or more anti-allergic agents can induce combinatorial or synergistic effects with respect to the release of histamine, leukotriene (34, prostaglandin E2, interleukin 4, interleukin 13, tumor necrosis factor α, or combinations thereof. For example, but not by way of limitation, synergistic decreases in the release of histamine, leukotriene (34, prostaglandin E2, interleukin 4, interleukin 13, tumor necrosis factor α, or combinations thereof from the target tissue and/or cell can occur when combinations of the disclosed composition and anti-allergic agents are administered.

In non-limiting embodiments, the disclosed methods can include administering a therapeutically effective amount of Tβ4, Tβ4 fragments, Tβ4 isoforms, Tβ4 derivatives, or variants thereof, wherein the therapeutically effective amount can decrease release of a pruritus-related factor or decrease release of an inflammatory cytokine from a target tissue for preventing or treating pruritus. In non-limiting embodiments, the therapeutically effective amount of Tβ4, Tβ4 fragments, Tβ4 isoforms, Tβ4 derivatives, or variants thereof in the pharmaceutical composition can be from about 50 μg/ml to about 10 mg/ml, from about 50 μg/ml to about 5 mg/ml, from about 50 μg/ml to about 1 mg/ml, or from about 100 μg/ml to about 1 mg/ml. The composition including the therapeutically effective amount of Tβ4, Tβ4 fragments, Tβ4 isoforms, Tβ4 derivatives, or variants thereof can decrease the release of the pruritus-related factor from the target tissue is reduced by at least about 10%, about 20%, about 30%, or about 40% after the administering the composition. The pruritus-related factor can include an allergic reaction factor (e.g., histamine, leukotriene (34, and prostaglandin E2), an inflammatory cytokine (e.g., interleukin 4, interleukin 13, and tumor necrosis factor α), or combinations thereof. In non-limiting embodiments, the composition can be applied to the target tissue in a form of a solution, lotion, plaster, gel, cream, paste, spray, suspension, dispersion, hydrogel, ointment, oil, patch, lens, inhaler, nasal spray, or foaming agent. The target tissue can include skin, connective tissue, nerve, soft tissue, epithelial tissue, or combinations thereof. In non-limiting embodiments, the composition can be applied to the target tissue at least one time a day. In non-limiting embodiments, the disclosed composition can be used alone or in combination with one or more agents, e.g., anti-allergic agents. Synergistic or combinatorial decreases in the release of histamine, leukotriene β4, prostaglandin E2, interleukin 4, interleukin 13, tumor necrosis factor α, or combinations thereof from the target tissue and/or cell can occur when combinations of the disclosed composition and anti-allergic agents are administered. Non-limiting exemplary anti-allergic agents can include anti-histamine agents, decongestants, steroids, mast cell stabilizers, leukotriene modifiers, nasal anticholinergics, epinephrine, immunomodulators, or combinations thereof.

In non-limiting embodiments, the disclosed method for treating pruritus can include administering a therapeutically effective amount of Tβ4, Tβ4 fragments, Tβ4 isoforms, Tβ4 derivatives, or variants thereof, wherein the therapeutically effective amount can decrease release of a pruritus-related factor or decrease release of an inflammatory cytokine from a target tissue, wherein the therapeutically effective amount of Tβ4, Tβ4 fragments, Tβ4 isoforms, Tβ4 derivatives, or variants thereof in the pharmaceutical composition can be from about 50 μg/ml to about 10 mg/ml, from about 50 μg/ml to about 5 mg/ml, from about 50 μg/ml to about 1 mg/ml, or alternately, from about 100 μg/ml to about 1 mg/ml, wherein the release of the pruritus-related factor from the target tissue can be reduced by at least about 10%, about 20%, about 30%, or about 40% after the administering the composition, wherein the pruritus-related factor can be selected from the group consisting of an allergic reaction factor, an inflammatory cytokine, or a combination thereof, wherein the allergic reaction factor can be selected from the group consisting of histamine, leukotriene (34, prostaglandin E2, and combinations thereof, wherein the inflammatory cytokine can be selected from the group consisting of interleukin 4, interleukin 13, tumor necrosis factor α, and combinations thereof, wherein the composition can be applied to the target tissue in a form of a solution, lotion, plaster, gel, cream, paste, spray, suspension, dispersion, hydrogel, ointment, oil, patch, lens, inhaler, nasal spray, or foaming agent, wherein the target tissue can include skin, connective tissue, nerve, soft tissue, epithelial tissue, or combinations thereof, wherein the epithelial tissue can be selected from the group consisting of an oral epithelial tissue, a pharyngeal epithelial tissue, a nasal epithelial tissue, an ocular epithelial tissue, a gastric epithelial tissue, and combinations thereof, and wherein the composition can be applied to the target tissue at least one time a day.

In non-limiting embodiments, the disclosed method for preventing pruritus can include administering a therapeutically effective amount of Tβ4, Tβ4 fragments, Tβ4 isoforms, Tβ4 derivatives, or variants thereof, wherein the therapeutically effective amount can decrease release of a pruritus-related factor or decrease release of an inflammatory cytokine from a target tissue, wherein the therapeutically effective amount of Tβ4, Tβ4 fragments, Tβ4 isoforms, Tβ4 derivatives, or variants thereof in the pharmaceutical composition can be from about 50 μg/ml to about 10 mg/ml, or alternately, from about 100 μg/ml to about 1 mg/ml, wherein the release of the pruritus-related factor from the target tissue can be reduced by at least about 10%, about 20%, about 30%, or about 40% after the administering the composition, wherein the pruritus-related factor can be selected from the group consisting of an allergic reaction factor, an inflammatory cytokine, or a combination thereof, wherein the allergic reaction factor can be selected from the group consisting of histamine, leukotriene (34, prostaglandin E2, and combinations thereof, wherein the inflammatory cytokine can be selected from the group consisting of interleukin 4, interleukin 13, tumor necrosis factor α, and combinations thereof, wherein the composition can be applied to the target tissue in a form of a solution, lotion, plaster, gel, cream, paste, spray, suspension, dispersion, hydrogel, ointment, oil, patch, lens, inhaler, nasal spray, or foaming agent, wherein the target tissue can include skin, connective tissue, nerve, soft tissue, epithelial tissue, or combinations thereof, wherein the epithelial tissue can be selected from the group consisting of an oral epithelial tissue, a pharyngeal epithelial tissue, a nasal epithelial tissue, an ocular epithelial tissue, a gastric epithelial tissue, and combinations thereof, and wherein the composition can be applied to the target tissue at least one time a day.

### EXAMPLES

The presently disclosed subject matter will be better understood by reference to the following Examples, which are provided as exemplary of the presently disclosed subject matter, and not by way of limitation.

### Example 1: Anti-Allergic Effects of Thymosin Beta 4

The objective of this study was to determine the effect of thymosin beta 4 (Tβ4) on pruritus treatment and prevention. The prevention and treatment effects of Tβ4 on pruritus were assessed using phorbol 12-myri state 13-acetate plus A23187 (PMACI)-induced human mast cell line (HMC-1) cells.

Materials:

Cell culture: Human Mast Cell Line (HMC-1) cells (ATCC, Manassas, Va., USA) were maintained in IMDM (Iscove's Modified Dulbecco's Media) medium (Thermo Fisher Scientific) containing 10% fetal bovine serum (FBS; Hyclone, GE Healthcare Life Sciences) and 1% penicillin-streptomycin (Sigma-Aldrich, St. Louis, Mo., USA) in a 37° C. humidified incubator with 5% CO2. The medium was changed every 2-3 days.

HMC-1 cell treatment: HMC-1 cells were seeded in 12-well plates at a density of 7×10⁵ cells per well and exposed to Tβ4 (1, 1.5, 3, and 6 μg/mL) for 1 hour before treatment with phorbol 12-myristate 13-acetate plus A23187 (PMACI; 30 nM of PMA plus 1.5 μM of A23187) to induce degranulation (activation) of mast cells and incubated at 37° C. After 2.5 hours, the medium was centrifuged (Hanil, UNION 32R PLUS) for 20 minutes at 1000×g. The supernate was transferred to another tube.

Methods:

Allergic reaction factors: Histamine (HIS), Leukotrien B4 (LTB4), and Prostaglandin E2 (PGE2) amounts were evaluated to determine the effects of the disclosed composition on the release of allergic reaction factors. Histamine levels were measured using the HIS (Histamine) ELISA kit (Elabscience, Huston, Tex., USA) on the supernate of the culture medium. 50 μL of the sample was added to 96-plate wells, then 50 μL of Biotinylated Detection Ab working solution was added and incubated at 37° C. for 45 minutes. 100 μL of HRP Conjugate working solution was added and incubated at 37° C. for 30 minutes. 90 μL of Substrate reagent was added and incubated at 37° C. for 15 minutes. Washing was performed between each step. 50 μL of stop solution was added without washing. Absorbance was measured at 450 nm with a spectrophotometer (Biotek, PowerWave XS2, Winooski, Vt., USA).

The leukotriene B4 amount was measured using the LTB4 (Leukotriene B4) ELISA kit (MybioSource, San Diego, Calif., USA) on the supernate of the culture medium. 50 μL of the sample was added to 96-plate wells, then 50 μL of Biotinylated Detection Ab working solution was added and incubated at 37° C. for 45 minutes. 100 μL of HRP Conjugate working solution was added and incubated at 37° C. for 30 minutes. 90 μL of Substrate reagent was added and incubated at 37° C. for 15 minutes. Washing was performed between each step. 50 μL of stop solution was added without washing. Absorbance was measured at 450 nm with a spectrophotometer (Biotek, PowerWave XS2, Winooski, Vt., USA).

The prostaglandin E2 (PGE2) amount was measured using a human PGE2 ELISA kit (Abeam, Cambridge, Mass.) on the supernate of the culture medium. 100 μL of the sample was added to 96-plate wells, then 50 μL of PGE2 Alkaline Phosphatase Conjugate was added. 50 ul of PGE2 Antibody was added and incubated on a shaker at room temperature for 45 minutes. 200 μL of pNPP substrate solution was added and incubated at 37° C. for 30 minutes. 90 μL of Substrate reagent was added and incubated at room temperature for 45 minutes. Washing was performed between each step. 50 μL of stop solution was added without washing. Absorbance was measured at 405 nm with a spectrophotometer (Biotek, PowerWave XS2, Winooski, Vt., USA).

Inflammatory cytokines: IL-4, IL-13, and TNF-α amounts were evaluated to determine the effects of the disclosed composition on the release of inflammatory cytokines. IL-4 and IL-13 amounts were measured using Human IL-4 (ab100570) and IL-13 (ab100553) ELISA kit (Abeam, Cambridge, Mass.) on the supernate of the culture medium. 100 μL of the sample was added to 96-plate wells, then 100 μL of Biotinylated IL-4 and IL-13 detection antibody were added and incubated at room temperature for 1 hour. 100 μL of HRP-streptavidin solution was added and incubated at room temperature for 45 minutes. 100 μL of TMB one step substrate reagent was added and incubated at room temperature for 30 minutes. Washing was performed between each step and incubated on a shaker. Finally, 50 μL of stop solution was added without washing. Absorbance was measured at 450 nm with a spectrophotometer (Biotek, PowerWave XS2, Winooski, Vt., USA).

The TNF-α amount was measured using a human TNF-alpha ELISA kit (Abeam, Cambridge, Mass.) on the supernate of the culture medium. 100 μL of the sample was added to 96-plate wells, then 50 μL of Biotinylated TNF alpha detection antibody was added and incubated at room temperature for 3 hours. 100 μL of HRP-streptavidin solution was added and incubated at room temperature for 30 minutes. 100 μL of Chromogen TMB substrate solution was added and incubated at room temperature of the light-shielding state for 20 minutes. Washing was performed between each step and incubated on a shaker. 50 μL of stop solution was added without washing. Absorbance was measured at 450 nm with a spectrophotometer (Biotek, PowerWave XS2, Winooski, Vt., USA).

Statistical analyses: The data were analyzed using the Student's t-test, and are presented as mean and SEM values. All samples were analyzed in three different experiments. The cutoff for statistical significance was set at P<0.05.

Results:

Allergic reaction factors: Mast cells are filled with granules containing various chemical mediators, including histamine, which induce allergic reactions by degranulation and release due to external stimuli. Therefore, to evaluate the inhibitory effect of Tβ4 on the degranulation of mast cells, HIS, LTB4, and PGE2 amounts were measured by ELISA.

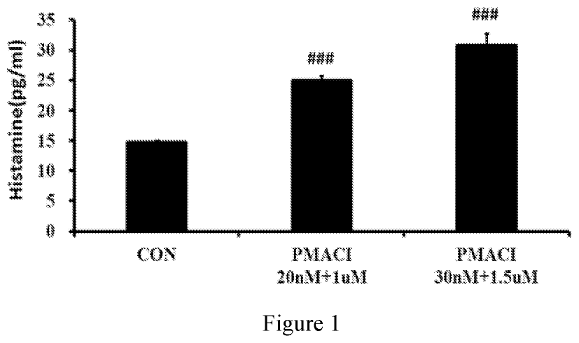

Determination of PMACI treatment concentration: As shown in FIG. 1, when the concentration of PMACI (PMA+CI) was 20 nM of PMA plus 1 μM of A23187 or 30 nM of PMA plus 1.5 μM of A23187, an increase in histamine release of 169% and 208% was observed when compared to the untreated normal control (CON). Therefore, the treatment concentration of PMACI (PMA+CI) in the subsequent experiments was determined to be 30 nM of PMA plus 1.5 μM of A23187, where secretion was twice that of the CON.

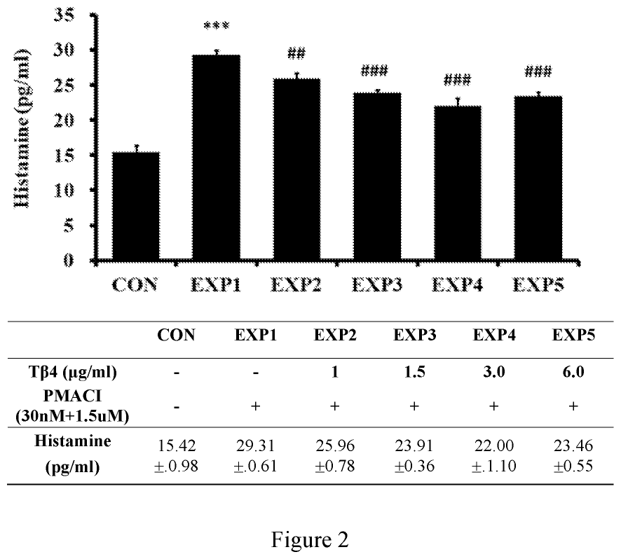

Histamine (HIS): As a result of evaluating the inhibitory effect on the release of histamine (HIS), a key substance causing pruritus, PMACI alone showed a significant increase in HIS release compared to normal (CON)(p<0.001). As shown in FIG. 2, when Tβ4 was used at a concentration of less than 1 μg/ml, there was no significant difference in the amount of HIS released compared to that of PMACI alone, but when Tβ4 was used at 1, 1.5, 3, 6 μg/ml, there was a significant inhibitory effect in the amount of HIS released compared to that of PMACI alone (p<0.01). Tβ4 at 3 μg/ml has the best effect of suppressing the increase of HIS release induced by PMACI treatment and can be expected to improve the pruritus symptoms.

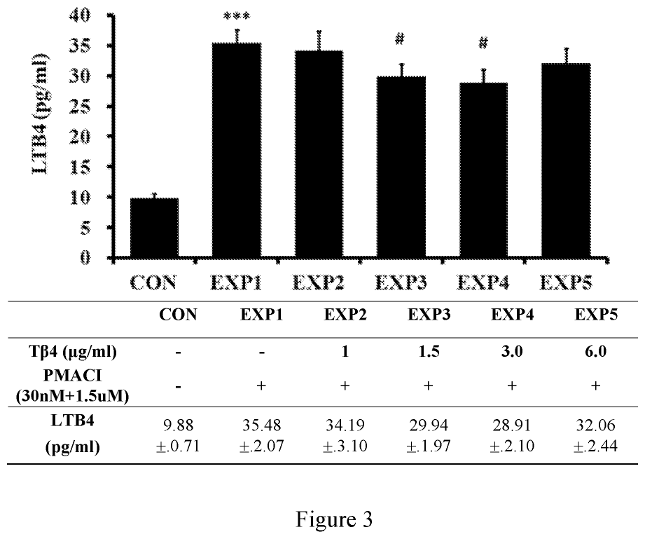

Leukotrien B4 (LTB4): As a result of evaluating the inhibitory effect on the release of Leukotrien B4 (LTB4), a key substance causing pruritus, PMACI alone showed a significant increase in the release of LTB4 compared to normal (CON) (p<0.001). As shown in FIG. 3, Tβ4 treatment at concentrations of 1.5 and 3 μg/ml showed a significant inhibitory effect in the amount of LTB4 released compared to that of PMACI alone (p<0.05). Tβ4 at 1.5 and 3 μg/ml are the most effective in inhibiting the increase of the release amount of LTB4 induced by PMACI treatment and can be expected to improve the effect of pruritus symptoms.

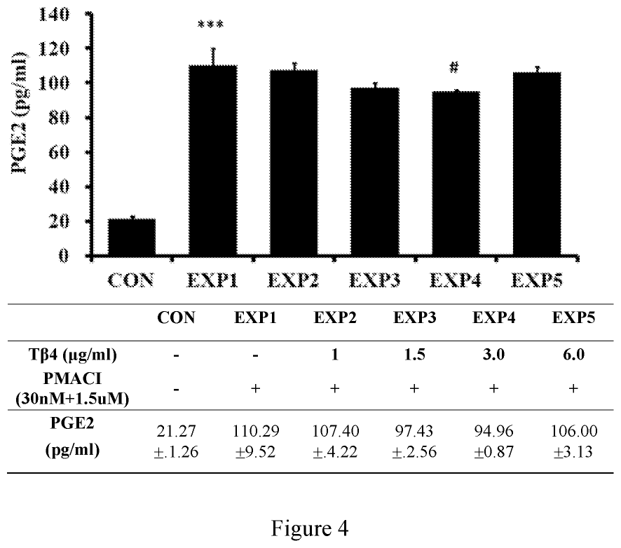

Prostaglandin E2 (PGE2): As a result of evaluating the inhibitory effect on the release of Prostaglandin E2 (PGE2), a key substance causing pruritus, PMACI alone showed a significant increase in the release of PGE2 compared to normal (CON) (p<0.001). As shown in FIG. 4, Tβ4 treatment at a concentration of 3 μg/ml showed a significant inhibitory effect in the amount of PGE2 released compared to that of PMACI alone (p<0.05). Tβ4 at 3 μg/ml is the most effective in inhibiting the increase of the release amount of PGE2 induced by PMACI treatment and can be expected to improve the effect of pruritus symptoms.

Inflammatory cytokines: Mast cells activated by PMACI release inflammatory cytokines. Therefore, evaluations of the inhibitory effects of Tβ4 on inflammatory cytokine release, including IL-4, IL-13, and TNF-α, were measured using ELISA.

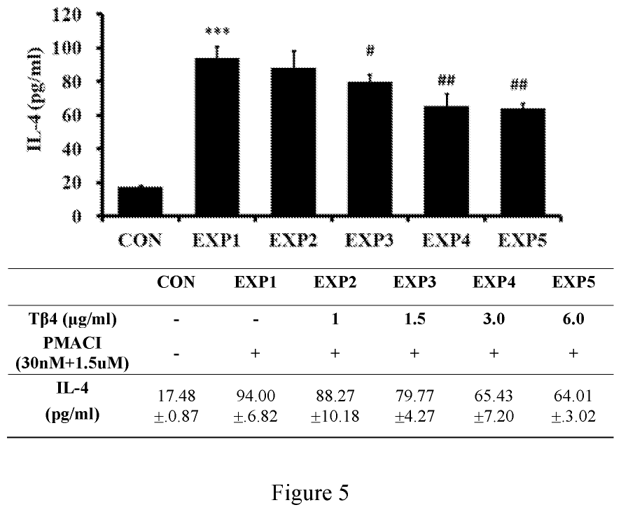

Interleukin-4 (IL-4): As a result of evaluating the inhibitory effect on the release of inflammatory cytokine factor Interleukin-4 (IL-4), PMACI alone showed a significant increase in the release of IL-4 compared to that of the untreated control (CON) (p<0.001). As shown in FIG. 5, Tβ4 at concentrations of 1.5, 3, and 6 μg/ml showed a significant inhibitory effect in reducing the amount of IL-4 released compared to that of PMACI alone (p<0.05). Tβ4 at above 1.5 μg/ml is effective in inhibiting the increase of the release of IL-4 induced by PMACI treatment and can be expected to improve the effect of pruritus symptoms.

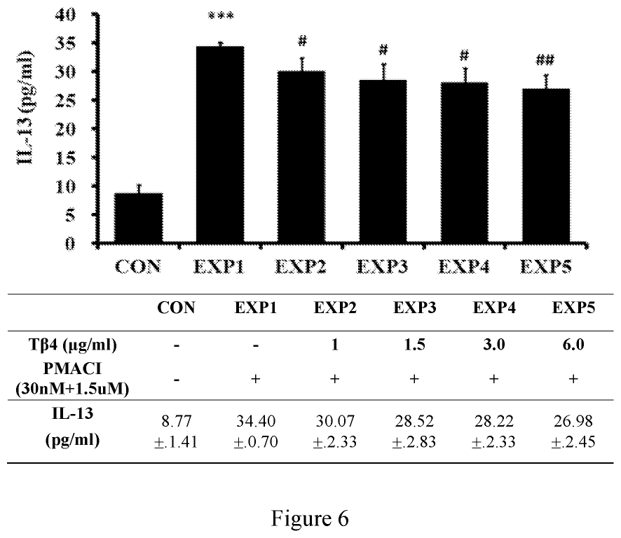

Interleukin-13 (IL-13): As a result of evaluating the inhibitory effect on the release of inflammatory cytokine factor Interleukin-13 (IL-13), PMACI alone showed a significant increase in the release of IL-13 compared to that of the untreated control (CON) (p<0.001). As shown in FIG. 6, Tβ4 at concentrations of 1, 1.5, 3, and 6 μg/ml showed a significant inhibitory effect in the amount of IL-13 released compared to that of PMACI alone (p<0.05). Tβ4 at or above 1 μg/ml concentration is effective in inhibiting the increase of the release amount of IL-13 induced by PMACI treatment and can be expected to improve the effect of pruritus symptoms.

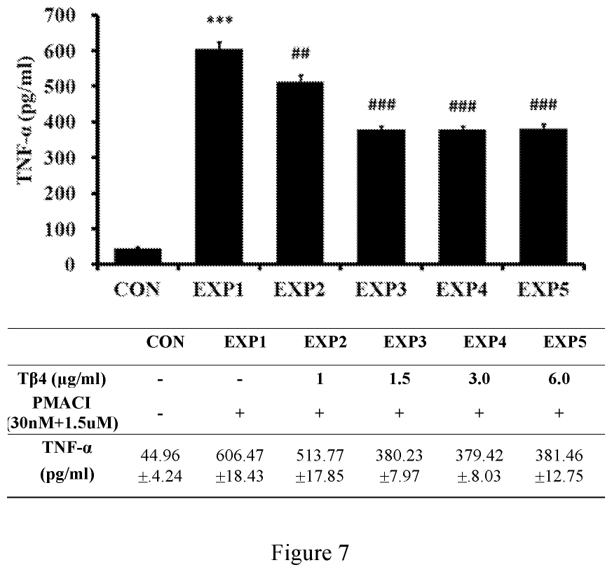

Tumor necrosis factor-alpha (TNF-α): As a result of evaluating the inhibitory effect on the release of inflammatory cytokine factor Tumor necrosis factor-alpha (TNF-α), PMACI alone showed a significant increase in the release of TNF-α compared to that of the untreated control (CON) (p<0.001). As shown in FIG. 7, Tβ4 at concentrations of 1, 1.5, 3, and 6 μg/ml showed a significant inhibitory effect in the amount of TNF-α released compared to that of PMACI alone (p<0.01). Tβ4 at 1 μg/ml and higher is effective in inhibiting the increase of the release amount of TNF-α induced by PMACI treatment and can be expected to improve the effect of pruritus symptoms.

Discussion:

The anti-allergic effects of Tβ4 on the symptoms of pruritus were evaluated by analyzing the release of PMACI-induced allergic factors in HMC-1.

The release levels of Histamine (HIS), Leukotrien B4 (LTB4), and Prostaglandin E2 (PGE2), which are the key substances causing pruritus, were significantly decreased when Tβ4 was used at a concentration of 1-6 μg/ml, compared with that of the PMACI treatment alone. In particular, when Tβ4 was used with a concentration of 3 μg/ml, release levels of HIS, LTB4, and PGE2 were decreased by 25%, 19%, and 14%, respectively, compared to that of the PMACI treatment alone. Based on this scientific evidence, Tβ4 has anti-allergic effects that significantly inhibit the degranulation of activated mast cells and show the best efficacy at 3 μg/ml.

TABLE 1
Inhibitory Effects of Pruritus-related Factors of thymosin Beta 4.

| | | Results | | | | | | |
|---|---|---|---|---|---|---|---|---|
| | | | EXP1 | EXP2 | EXP3 | EXP4 | EXP5 | |
| | Evaluation | | (PMACI | (Tβ4, | (Tβ4, | (Tβ4, | (Tβ4, | |
| Biomarkers | criteria | CON | only) | 1 μg/ml) | 1.5 μg/ml) | 3 μg/ml) | 6 μg/ml) | Remarks |
| Allergic | Histamine | 15.42 ± 0.98 | 29.31 ± 0.61 | 25.96 ± 0.78 | 23.91 ± 0.36 | 22.00 ± 1.10 | 23.46 ± 0.55 | Tβ4 (3 μg/ml) |
| reaction | | | | | | | | treatment |
| factors | | | | | | | | significantly |
| (pg/ml) | | | | | | | | reduced 24.9% |
| | | | | | | | | compared to |
| | | | | | | | | PMACI alone |
| | Leukotrien | 9.88 ± 0.71 | 35.48 ± 2.07 | 34.19 ± .3.10 | 29.94 ± 1.97 | 28.91 ± 2.10 | 32.06 ± 2.44 | Tβ4 (3 μg/ml) |
| | B4 | | | | | | | treatment |
| | | | | | | | | significantly |
| | | | | | | | | reduced 18.5% |
| | | | | | | | | compared to |
| | | | | | | | | PMACI alone |
| | PGE2 | 21.27 ± .1.26 | 110.29 ± 9.52 | 107.40 ± .4.22 | 97.43 ± 2.56 | 94.96 ± 0.87 | 106.00 ± 3.13 | Tβ4 (3 μg/ml) |
| | | | | | | | | treatment |
| | | | | | | | | significantly |
| | | | | | | | | reduced 13.9% |
| | | | | | | | | compared to |
| | | | | | | | | PMACI alone |
| Inflammatory | IL-4 | 17.48 ± 0.87 | 94.00 ± 6.82 | 88.27 ± 10.18 | 79.77 ± 4.27 | 65.43 ± 7.20 | 64.01 ± 3.02 | Tβ4 (6 μg/ml) |
| cytokines | | | | | | | | treatment |
| (pg/ml) | | | | | | | | significantly |
| | | | | | | | | reduced 31.9% |
| | | | | | | | | compared to |
| | | | | | | | | PMACI alone |
| | IL-13 | 8.77 ± 1.41 | 34.40 ± 0.70 | 30.07 ± 2.33 | 28.52 ± 2.83 | 28.22 ± 2.33 | 26.98 ± 2.45 | Tβ4 (6 μg/ml) |
| | | | | | | | | treatment |
| | | | | | | | | significantly |
| | | | | | | | | reduced 21.6% |
| | | | | | | | | compared to |
| | | | | | | | | PMACI alone |
| | TNF-α | 44.96 ± 4.24 | 606.47 ± 18.43 | 513.77 ± 17.85 | 380.23 ± 7.97 | 379.42 ± 8.03 | 381.46 ± 12.75 | Tβ4 (3 μg/ml) |
| | | | | | | | | treatment |
| | | | | | | | | significantly |
| | | | | | | | | reduced 37.4% |
| | | | | | | | | compared to |
| | | | | | | | | PMACI alone |

As shown in Table 1, when Tβ4 was treated at a concentration of 1-6 μg/ml, the release levels of inflammatory cytokine factors such as Interleukin-4 (IL-4), Interleukin-13 (IL-13), and Tumor necrosis factor-alpha (TNF-α) were significantly decreased compared with that of the PMACI treatment alone. In particular, when Tβ4 was treated at 6 μg/ml, the release levels of IL-4, IL-13, and TNF-α were decreased to 31.9%, 21.6%, and 37.1%, respectively, compared to that of the PMACI treatment alone.

Based on this scientific evidence, Tβ4 has an anti-allergic effect that significantly inhibits the release of inflammatory cytokines of activated mast cells and shows the best efficacy at or above 3 μg/ml concentration. A higher amount of Tβ4, Tβ4 fragments, Tβ4 isoforms, Tβ4 derivatives, or variants can be used for human applications to induce the clinically relevant anti-allergic effects. For example, Tβ4, Tβ4 fragments, Tβ4 isoforms, Tβ4 derivatives, or variants at a concentration range from about 50 μg/ml to about 10 mg/ml, or alternately, from about 100 μg/ml to about 1 mg/ml can be used for human subjects.

### REFERENCES

Shin Y W, Lee J O; pruritus, psychocutaneous disorder, neurocutaneous dermatoses. In Board. KTE(eds): Dermatology, 5th ed. p 93-104, Ryo Moon Gak, Seoul, 2008
Buddenkotte J, Steinhoff M. Pathophysiology and therapy of pruritus in allergic and atopic diseases. Allergy 2010; 65:805-21
Schmelz M, Schmidt R, Bickel A, Handwerker H O, Torebjork H E. Specific C-receptors for itch in human skin. J Neurosci 1997; 17:8003-8
Andrew D, Craig A D. Spinothalamic lamina I neurons selectively sensitive to histamine: a central neural pathway for itch. Nat Neurosci 2001; 4:72-7
Rivera J, Fierro N A, Olivera A, Suzuki R. New insights on mast cell activation via the high affinity receptor for IgE. Adv Immunol 2008; 98:85-120.
Moon T C, St Laurent C D, Morris K E, Marcet C, Yoshimura T, Sekar Y, et al. Advances in mast cell biology: new understanding of heterogeneity and function. Mucosal Immunol 2010; 3:111-28.
Blank U, Falcone F H, Nilsson G. The history of mast cell and basophil research—some lessons learnt from the last century. Allergy 2013; 68:1093-101.
LarchéM, Akdis C A, Valenta R. Immunological mechanisms of allergen-specific immunotherapy. Nat Rev Immunol. 2006 October; 6(10):761-71.
Steinhoff M, Buddenkotte J, Lerner E A6. Role of mast cells and basophils in pruritus. Immunol Rev. 2018 March; 282(1):248-264. doi: 10.1111/imr.12635.
Kawakami T, Galli S J. Regulation of mast-cell and basophil function and survival by IgE. Nat Rev Immunol 2002; 2:773-86.
Stone K D, Prussin C, Metcalfe D D. IgE, mast cells, basophils, and eosinophils. J Allergy Clin Immunol 2010; 125(2 Suppl 2):S73-80.
Theoharides T C, Alysandratos K D, Angelidou A, Delivanis D A, Sismanopoulos N, Zhang B, et al. Mast cells and inflammation. Biochim Biophys Acta 2012; 1822:21-33.
Bjorklund G, Dadar M, Aaseth J, Chirumbolo S. Thymosin (34: a multi-faceted tissue repair stimulating protein in heart injury. Curr Med Chem. 2019 Jul. 16. [Epub ahead of print]
Sosne G. Thymosin beta 4 and the eye: the journey from bench to bedside. Expert Opin Biol Ther. 2018 July; 18(supl):99-104.

All patents, patent applications, publications, product descriptions, and protocols cited in this specification are hereby incorporated by reference in their entireties. In case of a conflict in terminology, the present disclosure controls.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain using no more than routine experimentation, many equivalents to the specific embodiments described herein. Such equivalents are intended to be encompassed by the following claims.

Various publications and nucleic acid and amino acid sequence accession numbers are cited herein, the contents and full sequences of which are hereby incorporated by reference herein in their entireties.

1. A method for treating or preventing pruritus in a subject in need thereof, the method comprising administering a therapeutically effective amount of Tβ4, Tβ4 fragments, Tβ4 isoforms, Tβ4 derivatives, or variants thereof, wherein the therapeutically effective amount decreases release of a pruritus-related factor or decreases release of an inflammatory cytokine from a target tissue.

2. The method of claim 1, wherein the target tissue is selected from the group consisting of skin, connective tissue, nerve, soft tissue, epithelial tissue, and combinations thereof.

3. The method of claim 1, wherein the pruritus-related factor is an allergic reaction factor.

4. The method of claim 3, wherein the allergic reaction factor is selected from the group consisting of histamine, leukotriene β4, prostaglandin E2, and combinations thereof.

5. The method of claim 3, wherein the release of the allergic reaction factor from the target tissue is reduced by at least about 10%, about 20%, or about 30%.

6. The method of claim 1, wherein the inflammatory cytokine is selected from the group consisting of interleukin 4, interleukin 13, tumor necrosis factor α, and combinations thereof.

7. The method of claim 6, wherein the release of the inflammatory cytokine from the target tissue is reduced by at least about 10%, about 20%, about 30%, or about 40%.

8. The method of claim 1, wherein the therapeutically effective amount of Tβ4, Tβ4 fragments, Tβ4 isoforms, Tβ4 derivatives, or variants thereof is applied to the target tissue in a form of a solution, lotion, plaster, gel, cream, paste, spray, suspension, dispersion, hydrogel, ointment, oil, patch, lens, inhaler, nasal spray, or foaming agent.

9. The method of claim 1, wherein the therapeutically effective amount of Tβ4, Tβ4 fragments, Tβ4 isoforms, Tβ4 derivatives, or variants thereof is applied to the target tissue at least one time a day.

10. The method of claim 1, wherein the therapeutically effective amount of Tβ4, Tβ4 fragments, Tβ4 isoforms, Tβ4 derivatives, or variants thereof is from about 50 μg/ml to about 10 mg/ml.

11. The method of claim 2, wherein the epithelial tissue is selected from the group consisting of an oral epithelial tissue, a pharyngeal epithelial tissue, a nasal epithelial tissue, an ocular epithelial tissue, a gastric epithelial tissue, and combinations thereof.

12. A pharmaceutical composition for treating or preventing pruritus in a subject comprising a therapeutically effective amount of Tβ4, Tβ4 fragments, Tβ4 isoforms, Tβ4 derivatives, or variants thereof, wherein the therapeutically effective amount is present in an amount to decrease release of a pruritus-related factor or decrease release of an inflammatory cytokine from a target tissue.

13. The pharmaceutical composition of claim 12, wherein the pruritus-related factor is an allergic reaction factor.

14. The pharmaceutical composition of claim 13, wherein the allergic reaction factor is selected from the group consisting of histamine, leukotriene β4, prostaglandin E2, and combinations thereof.

15. The pharmaceutical composition of claim 12, wherein the inflammatory cytokine is selected from the group consisting of interleukin 4, interleukin 13, tumor necrosis factor α, and combinations thereof.

16. The pharmaceutical composition of claim 12, wherein the therapeutically effective amount of Tβ4, Tβ4 fragments, Tβ4 isoforms, Tβ4 derivatives, or variants thereof in the composition is from about 50 μg/ml to about 10 mg/ml.

17. The pharmaceutical composition of claim 12, wherein the pharmaceutical composition is in a form of a solution, lotion, plaster, gel, cream, paste, spray, suspension, dispersion, hydrogel, ointment, oil, patch, lens, inhaler, nasal spray, or foaming agent.
